(19) United States
(12) Patent Application Publication
(10) Pub. No.: US 2026/0216029 A1
(43) Pub. Date: Jul. 30, 2026

(54) MAKEUP COMPOSITION FOR NEEDLE COSMETIC INSTRUMENT, COSMETIC KIT, AND COSMETIC METHOD

(71) Applicant: SHISEIDO COMPANY, LTD., Chuo-ku, Tokyo, (JP)

(72) Inventors: Shuting LIN, Tokyo (JP); Hiroko SHIMIZU, Tokyo (JP)

(73) Assignee: SHISEIDO COMPANY, LTD., Chuo-ku, Tokyo, (JP)

(21) Appl. No.: 19/147,000

(22) PCT Filed: Jan. 26, 2024
(86) PCT No.: PCT/JP2024/002471

§ 371 Date: Jul. 10, 2025

(30) Foreign Application Priority Data
Feb. 10, 2023 (JP) 2023-019229

(51) Int. Cl. A61K 8/44; A61M 5/32; A61Q 19/00

(52) U.S. Cl. CPC A61K 8/442; A61M 5/3286; A61Q 19/00; A61K 2800/20; A61K 2800/87

(57) ABSTRACT

Provided is a cosmetic composition for needle-type cosmetic instruments that has high penetration. A cosmetic composition for needle-type cosmetic instruments, wherein a dynamic surface tension at a temperature of 25±1° C., measured by Wilhelmy method, is 50 mN/m or less.

### FIELD

The present invention relates to a cosmetic composition for needle-type cosmetic instruments. Further, the present invention relates to a cosmetic kit comprising the cosmetic composition for needle-type cosmetic instruments and a cosmetic method using the cosmetic composition for needle-type cosmetic instruments and/or the cosmetic kit.

### BACKGROUND

As a needle-type cosmetic instrument, for example, a multi-needle plate of a syringe comprising a plurality of needles (multi-needle, injection needle mechanism) is known.

PTL 1 reports a needle-like projection-including part that is able to retain a liquid composition/liquid cosmetic, the active component of which is not limited, on the surface and the inside of a target object so as not to leak from a gap. In addition, the needle-like projection-including part of PTL 1 can be used by being mounted to a needle-fixable injection device as a needle-type cosmetic instrument.

### CITATION LIST

### Patent Literature

[PTL 1] WO 2022/071322

### SUMMARY

### Technical Problem

In terms of penetration of cosmetic compositions for needle-type cosmetic instruments to date, there remains room for improvement.

The present invention aims to improve the above circumstances, and an object thereof is to provide a cosmetic composition for needle-type cosmetic instruments that has high penetration.

The present invention also provides a kit comprising the cosmetic composition for needle-type cosmetic instruments that has high penetration and a cosmetic method using the same.

### Solution to Problem

The present invention that achieves the above object is as follows.

### Aspect 1

A cosmetic composition for needle-type cosmetic instruments, wherein a dynamic surface tension at a temperature of 25±1° C., measured by Wilhelmy method, is 50 mN/m or less.

### Aspect 2

The composition according to Aspect 1, comprising a dynamic surface tension-reducing component selected from a group consisting of amino acid-based substances, surfactants, lower alcohols, higher alcohols, polyhydric alcohols, aromatic alcohols, silicones, fluorine compounds, saccharides, terpenes, acrylic compounds, polyethylene glycol, polyvinyl alcohol, and combinations thereof.

### Aspect 3

The composition according to Aspect 2, wherein content of the dynamic surface tension-reducing component is an amount that reduces the dynamic surface tension by 20 mN/m or more.

### Aspect 4

The composition according to Aspect 2 or 3, wherein the amino acid-based substance comprises sodium dilauroyl glutamate lysine and/or potassium cocoyl glutamate.

### Aspect 5

A cosmetic kit comprising a needle-type cosmetic instrument and the cosmetic composition for needle-type cosmetic instruments according to any one of Aspects 1 to 4.

### Aspect 6

A cosmetic method using a needle-type cosmetic instrument and the cosmetic composition for needle-type cosmetic instruments according to any one of Aspects 1 to 4.

### Aspect 7

The cosmetic composition for needle-type cosmetic instruments according to any one of Aspects 1 to 4, the kit according to Aspect 5, or the method according to Aspect 6, wherein the needle-type cosmetic instrument comprises a needle-like projection-including part that injects the cosmetic composition for needle-type cosmetic instruments into a target object.

### Advantageous Effects of Invention

According to the present invention, a cosmetic composition for needle-type cosmetic instruments that has high penetration can be provided.

According to the present invention, a kit comprising the above cosmetic composition for needle-type cosmetic instruments that has high penetration and a cosmetic method using the same can also be provided.

### BRIEF DESCRIPTION OF DRAWINGS

FIG. 1 is a schematic view showing one embodiment of the needle-like projection-including part according to the present invention.

FIG. 2 is a schematic view showing a portion of another embodiment of the needle-like projection-including part according to the present invention.

FIG. 3 is a schematic view showing one embodiment of a cosmetic instrument comprising a needle-like projection-including part and a fixing device.

FIG. 4 is a chart showing penetration improvement rates of Examples 1 and 2 and Comparative Examples 1, 3, and 4 with respect to Comparative Example 2.

FIG. 5 is a chart showing penetration improvement rates of Examples 3 and 4 and Comparative Examples 1 and 5 with respect to Comparative Example 2.

### DESCRIPTION OF EMBODIMENTS

Hereinafter, embodiments of the present invention will be described in detail. The present invention is not limited to the following embodiments, and various modifications can be made within the scope of the invention.

### <<Cosmetic Composition for Needle-Type Cosmetic Instruments>>

The cosmetic composition for needle-type cosmetic instruments of the present invention (hereinafter, also simply referred to as “composition of the present invention”) is a cosmetic composition for needle-type cosmetic instruments, wherein a dynamic surface tension at a temperature of 25±1° C., measured by the Wilhelmy method, is 50 mN/m or less.

The present inventors have carried out various studies in order to improve penetration of cosmetic compositions for needle-type cosmetic instruments into the skin, and as a result, have discovered that penetration of the composition of the present invention can be improved by adjusting the dynamic surface tension thereof at a temperature of 25±1° C., measured by the Wilhelmy method, to 50 mN/m or less.

Since the composition of the present invention is for use in needle-type cosmetic instruments, the penetration thereof can be improved when applied to the skin via a needle-type cosmetic instrument. Applying the composition of the present invention to the skin via a needle-type cosmetic instrument refers to bringing the composition of the present invention into contact with the skin at any point in time from when a needle of the needle-type cosmetic instrument begins to puncture the skin to immediately after the needle is withdrawn from the skin. In particular, it is preferable that the composition of the present invention be brought into contact with the skin for a certain period of time (for example, about 1 second to 30 minutes) in a state in which a needle of the needle-type cosmetic instrument punctures the skin. In addition, the needle-type cosmetic instrument is not particularly limited and, for example, may be a cosmetic instrument as disclosed in PTL 1.

In the present invention, the dynamic surface tension at a temperature of 25±1° C., measured by the Wilhelmy method, is also simply referred to as “dynamic surface tension”. In addition, the dynamic surface tension of the composition of the present invention can be determined by measuring at a temperature of 25±1° C., based on the Wilhelmy method, using a DCAT21 device manufactured by DataPhysics and carrying out an analysis with the SCAT software accompanying the device.

The dynamic surface tension of the composition of the present invention, more specifically, for example, may be 50 mN/m or less, 49 mN/m or less, 48 mN/m or less, 47 mN/m or less, 46 mN/m or less, 45 mN/m or less, 44 mN/m or less, 43 mN/m or less, 42 mN/m or less, 41 mN/m or less, 40 mN/m or less, 39 mN/m or less, 38 mN/m or less, 37 mN/m or less, 36 mN/m or less, 35 mN/m or less, 34 mN/m or less, 33 mN/m or less, 32 mN/m or less, 31 mN/m or less, or 30 mN/m or less. In addition, the lower limit value of dynamic surface tension of the composition of the present invention is not particularly limited and, for example, may be 1.0 mN/m or more, 5.0 mN/m or more, 10 mN/m or more, 12 mN/m or more, 14 mN/m or more, 16 mN/m or more, 18 mN/m or more, 20 mN/m or more, or 22 mN/m or more.

In the present invention, the method for adjusting the dynamic surface tension of the composition is not particularly limited and, for example, can be adjusted by adding a component that can affect dynamic surface tension and/or increasing or decreasing the addition amount thereof. As the component that can affect the dynamic surface tension of the composition, for example, a component that lowers dynamic surface tension, a component that raises dynamic surface tension, or a combination of these components can be used. However, the composition of the present invention preferably comprises a component that lowers dynamic surface tension, from the viewpoint of facilitating adjustment of dynamic surface tension to 50 mN/m or less.

In the following, each component that can be contained in the composition of the present invention will be described by way of example. However, the present invention is not limited to these components. In addition, the “component that lowers dynamic surface tension” described below is also simply referred to as a “dynamic surface tension-reducing component”.

### (Dynamic Surface Tension-Reducing Component)

The composition of the present invention preferably comprises a dynamic surface tension-reducing component. In particular, the composition of the present invention preferably contains a dynamic surface tension-reducing component selected from the group consisting of amino acid-based substances, surfactants, lower alcohols (referring to monohydric alcohols having 5 or fewer carbon atoms, but excluding alcohols such as aromatic alcohols recited below), higher alcohols (referring to monohydric alcohols having 6 or more carbon atoms, but excluding alcohols such as aromatic alcohols recited below), polyhydric alcohols, aromatic alcohols, silicones, fluorine compounds, saccharides, terpenes, acrylic compounds, polyethylene glycol, polyvinyl alcohol, and combinations thereof.

In the present invention, specific examples of the dynamic surface tension-reducing component will be described by way of example below, but are not limited thereto.

The amino acid-based substance as the dynamic surface tension-reducing component may comprise a salt of an acylamino acid, for example, may comprise at least one selected from the group consisting of sodium dilauroyl glutamate lysine, potassium cocoyl glutamate, sodium cocoyl glycinate, arginine cocoate, and sodium pyrrolidone carboxylate, and in particular, preferably comprises sodium dilauroyl glutamate lysine and/or potassium cocoyl glutamate. Sodium dilauroyl glutamate lysine and potassium cocoyl glutamate are generally used as surfactants, but in the present invention, are mainly used as components for lowering the dynamic surface tension of the composition of the present invention.

The surfactant as the dynamic surface tension-reducing component may comprise at least one selected from the group consisting of sorbitan sesquiisostearate, PEG-30 phytosterol, PEG-60 hydrogenated castor oil, and polyglyceryl-2 diisostearate.

The lower alcohol as the dynamic surface tension-reducing component may comprise at least one selected from the group consisting of ethanol, methanol, and 1,3-butylene glycol. The polyhydric alcohol as the dynamic surface tension-reducing component may comprise at least one selected from the group consisting of dipropylene glycol and glycerin. The aromatic alcohol as the dynamic surface tension-reducing component may comprise phenoxyethanol.

The terpene as the dynamic surface tension-reducing component may comprise dipotassium glycyrrhizate.

In the present invention, it is preferable that the content of the dynamic surface tension-reducing component be an amount that reduces the dynamic surface tension by 20 mN/m or more. The description “the content of the dynamic surface tension-reducing component be an amount that reduces the dynamic surface tension by 20 mN/m or more” means an amount that reduces the dynamic surface tension of the composition of the present invention by 20 mN/m or more compared to the case where the dynamic surface tension-reducing component is not contained. The content of the dynamic surface tension-reducing component may be an amount that reduces the dynamic surface tension by 20 mN/m or more, by 30 mN/m or more, by 40 mN/m or more, or by 45 mN/m or more.

In the present invention, the specific content of the dynamic surface tension-reducing component is not particularly limited. For example, the content by type of the dynamic surface tension-reducing components may each be 0.010% by mass or greater, 0.015% by mass or greater, 0.020% by mass or greater, 0.025% by mass or greater, 0.028% by mass or greater, 0.030% by mass or greater, 0.035% by mass or greater, 0.040% by mass or greater, 0.045% by mass or greater, 0.050% by mass or greater, 0.055% by mass or greater, 0.060% by mass or greater, 0.065% by mass or greater, 0.070% by mass or greater, 0.075% by mass or greater, 0.080% by mass or greater, 0.085% by mass or greater, 0.090% by mass or greater, 0.095% by mass or greater, 0.10% by mass or greater, 0.15% by mass or greater, 0.20% by mass or greater, 0.25% by mass or greater, or 0.30% by mass or greater, and may each be 5.0% by mass or less, 4.0% by mass or less, 3.0% by mass or less, 2.0% by mass or less, 1.0% by mass or less, 0.9% by mass or less, 0.8% by mass or less, 0.7% by mass or less, 0.6% by mass or less, or 0.5% by mass or less, relative to the entire composition. The “content by type” refers to, for example, if only one amino acid-based substance is contained, the content thereof, or if two or more amino acid-based substances are contained, the total content thereof. In addition, the content by type of the dynamic surface tension-reducing components may be appropriately adjusted in accordance with the degree to which the dynamic surface tension of the entire composition is affected.

### (Water)

The composition of the present invention can comprise water. In particular, water may be blended in the composition of the present invention, as needed, in accordance with the intended formulation, for example, an aqueous system (including an aqueous dispersion system), a solubilized system, an emulsified system, a water-oil bilayer system, or a water-oil-powder trilayer system.

The water is not particularly limited and, for example, may be water used in cosmetics and quasi-drugs. For example, ion-exchanged water, distilled water, ultrapure water, and tap water can be used.

The content of water, if comprising any, is not particularly limited and may be appropriately adjusted in accordance with the intended formulation and the contents of other components.

### (Additional Optional Component)

Examples of an additional optional component that can be contained in the composition of the present invention include, but are not limited to, amino acid-based substances other than the amino acid-based substances described above, cosmetic substances, lower alcohol components other than the lower alcohol components described above, powder components, liquid oils and fats, solid oils and fats, waxes, hydrocarbon oils, higher fatty acids, higher alcohols, ester oils, silicone oils, anionic surfactants, cationic surfactants, amphoteric surfactants, nonionic surfactants, water-soluble polymers, thickeners, film-forming agents, ultraviolet absorbers, sequestering agents, saccharide organic amines, polymer emulsions, pH adjusters, skin nutrients, antioxidants, antioxidant aids, and fragrances.

In the following, the cosmetic substance that is one of the optional components will be described by way of example.

The composition of the present invention can comprise a cosmetic substance. The cosmetic substance refers to a substance that imparts some cosmetic effect (for example, a moisturizing effect, a firming effect, a barrier function-improving effect, a blood flow-enhancing effect, a whitening effect, an anti-spot effect, an anti-wrinkle effect, an anti-acne effect, or an anti-inflammatory effect) to the skin. Therefore, the composition of the present invention may be a moisturizer, a firming agent, a barrier function-improving agent, a blood flow enhancer, a whitening agent, an anti-spot agent, an anti-wrinkle agent, an anti-acne agent, or an anti-inflammatory agent. Among these cosmetic substances, in particular, water-soluble cosmetic substances are preferable.

In the present invention, the cosmetic substance is not particularly limited, and examples include alkoxysalicylic acid and salts thereof, 1-piperidinepropionic acid and salts thereof, tranexamic acid and salts thereof, hyaluronic acid and salts thereof, vitamin A group and derivatives thereof, vitamin B group and derivatives thereof, vitamin C group and derivatives thereof, alkylene oxide derivatives, proteins and peptides such as collagen and EGF, amino acids, and polyhydric alcohols such as glycerin. The composition of the present invention may comprise these cosmetic substances individually or in combination of two or more. Among these cosmetic substances, those that are used as dynamic surface tension-reducing components are preferably used.

Alkoxysalicylic acid is a compound in which a hydrogen atom at any of position 3, position 4, and position 5 of salicylic acid is substituted with an alkoxy group. The alkoxy group as a substituent is preferably any of a methoxy group, an ethoxy group, a propoxy group, an isopropoxy group, a butoxy group, or an isobutoxy group, and more preferably a methoxy group or an ethoxy group. Specific compounds as examples of alkoxysalicylic acid include 3-methoxysalicylic acid. In addition, salts of alkoxysalicylic acid are not particularly limited, and examples include alkali metal salts and alkaline earth metal salts such as sodium salts, potassium salts, and calcium salts.

1-Piperidinepropionic acid is the compound shown in the following chemical formula:

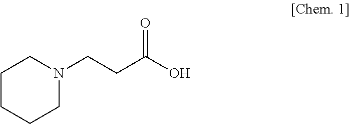 [SMILES: O=C(O)CCN1CCCCC1]

Salts of 1-piperidinepropionic acid are not particularly limited, and examples include alkali metal salts and alkaline earth metal salts such as sodium salts, potassium salts, and calcium salts; and other salts such as ammonium salts and amino acid salts.

Salts of tranexamic acid are not particularly limited, and examples include alkali metal salts and alkaline earth metal salts such as sodium salts, potassium salts, and calcium salts.

Salts of hyaluronic acid are not particularly limited, and examples include alkali metal salts and alkaline earth metal salts such as sodium salts, potassium salts, and calcium salts.

Vitamin A group and derivatives thereof are not particularly limited, and examples include retinol.

Vitamin B group and derivatives thereof are not particularly limited, and examples include vitamin B1, vitamin B2, vitamin B3 (also referred to as “niacin”), vitamin B5, vitamin B6, and vitamin B12, and derivatives thereof. Note that “niacin” includes nicotinic acid and niacinamide (also referred to as “nicotinamide”).

Vitamin C group and derivatives thereof are not particularly limited, and examples include vitamin C, vitamin C glucoside, and ethyl vitamin C.

Alkylene oxide derivatives can be represented by the following general formula (I).

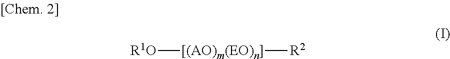

wherein AO is an oxyalkylene group having 3 or 4 carbon atoms, EO is an oxyethylene group, m and n are average numbers of moles added for the oxyalkylene group and the oxyethylene group, respectively, 1≤m≤70, and 1≤n≤70. The ratio of oxyethylene groups relative to the total of oxyalkylene groups having 3 or 4 carbon atoms and oxyethylene groups is 20 to 80% by weight. The oxyalkylene group having 3 or 4 carbon atoms and the oxyethylene group may be added in a block or random manner. R¹ and R² may be the same or different and are a hydrocarbon group having 1 to 4 carbon atoms or a hydrogen atom, and the ratio of the number of hydrogen atoms relative to the number of hydrocarbon groups in R¹ and R² is 0.15 or less.

In addition to those described above, examples of cosmetic substances include, but are not limited to, arbutin, fat cells as a filler, and botulinum toxin (Botox, Btx) in wrinkle treatments.

In the composition of the present invention, the content of the cosmetic substance, if comprising any, is not particularly limited and, for example, may be 0.001% by mass or greater, 0.005% by mass or greater, 0.01% by mass or greater, 0.05% by mass or greater, 0.1% by mass or greater, 0.5% by mass or greater, 1.0% by mass or greater, 2.0% by mass or greater, 3.0% by mass or greater, 4.0% by mass or greater, or 5.0% by mass or greater, and may be 30% by mass or less, 25% by mass or less, 20% by mass or less, 15% by mass or less, or 10% by mass or less, relative to the entire composition.

In addition to the components described above, the composition of the present invention may further comprise, for example, a cell suspension, a gel-like material, a therapeutic substance (for example, an antibiotic, an anesthetic, an analgesic, a vaccine, or an antibody), or a diagnostic substance.

### <<Cosmetic Kit>>

The present invention also provides a cosmetic kit. The cosmetic kit of the present invention is
a cosmetic kit comprising a needle-type cosmetic instrument and a cosmetic composition for needle-type cosmetic instruments,
wherein the cosmetic composition for needle-type cosmetic instruments has a dynamic surface tension at a temperature of 25±1° C., measured by the Wilhelmy method, of 50 mN/m or less.

The “cosmetic composition for needle-type cosmetic instruments” contained in the cosmetic kit of the present invention may be the same as the composition of the present invention described above, and thus the description thereof is omitted.

The “needle-type cosmetic instrument” contained in the cosmetic kit of the present invention is not particularly limited and, for example, the cosmetic instrument (device) disclosed in PTL 1 is preferably used.

The needle-type cosmetic instrument used in the present invention may comprise a needle-like projection-including part that injects the composition of the present invention into a target object.

More specifically, the needle-type cosmetic instrument of the present invention, for example, may be a cosmetic instrument comprising a needle-like projection-including part that injects a composition of the present invention into a target object,
wherein the needle-like projection-including part comprises
a plate in which a discharge hole that penetrates vertically is formed;
one or a plurality of fine needle-like projections protruding from one face of the plate; and
a lip portion formed on an outer edge portion of one face of the plate, and
wherein, when the composition of the present invention is pushed out from the discharge hole,
   the composition of the present invention spreads within a space formed between the target object,
   the lip portion, and one face of the plate, and the composition of the present invention enters into
   the target object via a hole opened by the one or plurality of needle-like projections, or
may be a cosmetic instrument comprising a needle-like projection-including part that injects the composition of the present invention into a target object,
wherein the needle-like projection-including part comprises
a plate;
one or a plurality of base portions protruding from one face of the plate;
one or a plurality of fine needle-like projections protruding from a front-end face of the one or each of the plurality of base portions; and
a lip portion formed on an outer edge portion of a front-end face of a base portion,
wherein in the plate and the one or plurality of base portions, a discharge hole penetrating vertically from the other face of the plate to the front-end face of the base portion is formed, and
wherein, when the composition of the present invention is discharged from the discharge hole, the composition of the present invention spreads within a space formed between the target object, the lip portion, and the front-end face of the base portion, and the composition of the present invention enters into the target object via a hole opened by the one or plurality of needle-like projections.

In the present invention, the needle-like projection-including part has a function of injecting the composition of the present invention into a target object. The target object may be the skin of a human, an animal, or a plant.

In the following, the needle-like projection-including part contained in a needle-type cosmetic instrument of the present invention will be described by way of example using the drawings.

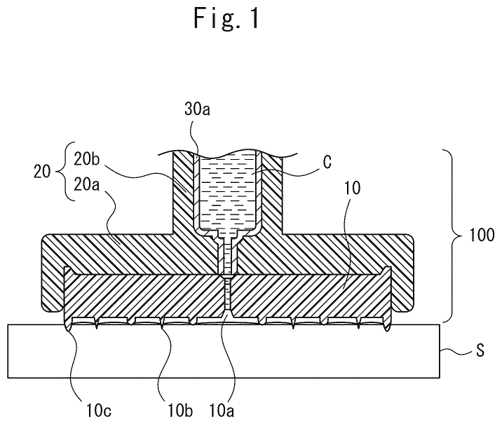

FIG. 1 is a schematic view showing one embodiment of the needle-like projection-including part according to the present invention.

FIG. 1 shows a state in which the needle-like projection-including part 100 according to the present invention is puncturing a target object (skin) S. As shown in FIG. 1, the needle-like projection-including part 100 comprises a plate 10 and an attaching frame 20. In addition, the plate 10 comprises a discharge hole 10a that penetrates vertically; a plurality of fine needle-like projections 10b that protrude from one face of the plate; and a lip portion 10c formed on an outer edge portion of one face of the plate.

In FIG. 1, the plate 10 comprises a plurality of fine needle-like projections 10b. However, in the present invention, there may be only one such fine needle-like projection 10b. In addition, in the present invention, such a fine needle-like projection (also referred to a “fine needle” or a “microneedle”) needs only to exhibit a certain degree of hardness so as to pierce the skin and cause stimulation, and the constituting material itself is not particularly limited. However, the fine needle-like projection is to be attached to the skin for an extended period of time, and is thus required to be made of a biocompatible material, and may preferably be formed of a material that can dissolve or swell in vivo. Such material includes, for example, polyglycolic acid (PGA), polylactic acid (PLA), poly lactic-co-glycolic acid copolymer (PLGA); polysaccharides such as hyaluronic acid, chitosan, maltose, alginate, amylose, and agarose; celluloses such as carboxymethyl cellulose and hydroxypropyl cellulose; and starches, and may be formed of a single material or formed from a mixture in which two or more materials are appropriately blended. Among these materials, in particular, polyglycolic acid (PGA), polylactic acid (PLA), or poly lactic-co-glycolic acid copolymer (PLGA) is preferably included. Alternatively, the fine needle-like projection may be formed of a resin that does not dissolve or swell in vivo and has no biological effect, and more specifically, may be formed of, for example, polymethyl methacrylate, cellulose acetate, ethyl cellulose, polyethylene resin, polypropylene resin, ethylene-propylene copolymer, ethylene-vinyl acetate copolymer, vinyl chloride-based resin, vinylidene chloride resin, vinyl acetate-vinyl chloride copolymer, polyamide-based resin, polyester resin, ABS resin, SIS resin, SEBS resin, urethane resin, silicone resin, stainless steel, aluminum, or magnesium.

As shown in FIG. 1, when the composition C of the present invention is pushed out from the discharge hole 10a, the composition C of the present invention spreads within a space formed between the target object S, the lip portion 10c, and one face of the plate 10, and the composition C of the present invention enters into the target object S via holes opened by the plurality of needle-like projections 10b, i.e., can penetrate from gaps between surfaces of the needle-like projections 10b and the target object S. The composition C of the present invention may be directly stored in the attaching barrel 20b, or, for example, may be stored in the syringe barrel (not illustrated) and the syringe barrel tip 30a via the syringe. In FIG. 1, the syringe barrel tip 30a is a part of the syringe, and the entire syringe is not shown. The method for pushing out the composition C of the present invention is not particularly limited and, for example, when the needle-like projection-including part 100 is attached to a syringe, can be carried out by the pusher of the syringe. After injection, the state in which the needle-like projection-including part 100 is pressed against the target object S via the syringe may be maintained for a predetermined period of time (less than 1 second to about 30 minutes, preferably from several seconds to several minutes). After the predetermined time has elapsed, the needle-like projections 10b may be withdrawn from the target object S by retracting the syringe away from the target object S. As such, in the present invention, the composition C of the present invention can be spread over the target object S on the inside of the lip portion 10c while penetrating into the inside of the target object S from holes opened by the needle-like projections 10b. Therefore, the composition C of the present invention can penetrate from both the outside and inside of the target object S. For example, in the case where the target object S is human skin and the composition C of the present invention comprises a cosmetic substance, the cosmetic substance can penetrate from both the outside and inside of the skin.

Although not shown in FIG. 1, the needle-like projection-including part 100 may integrally comprise a plate 10 and an attaching frame 20. Specifically, the needle-like projection-including part 100 may be of an integrated type. The integrated needle-like projection-including part 100, for example, may be composed of a biocompatible material, which includes biodegradable resins such as polyglycolic acid, polylactic acid, and polyglycolic acid-polylactic acid copolymer. More specifically, the integrated needle-like projection-including part 100, for example, may be composed of a thermoplastic resin such as polypropylene (PP), polyethylene (PE), acrylonitrile-butadiene-styrene (ABS), polybutylene terephthalate (PBS), polyacetal (POM), polyethylene terephthalate (PET), polycarbonate (PC), or polyether ether ketone (PEEC).

Although FIG. 1 illustrates an example in which there is one central hole as the discharge hole 10a, there may not be only one hole, i.e., may be two or more. The hole may be central or may not be central. Although the needle-like projections 10b are exemplified as having a conical outer shape, the needle-like projections may be polygonal pyramid-shaped or other needle-like shapes (such as star-type or cross-type). In addition, hollow needle-like projections can be used in place of solid needle-like projections. Further, the solid needle-like projections constituting the needle-like micro-projections may have a diameter, at the thickest portion of the needles, in the range of 2 to 5000 μm, and more preferably in the range of 5 to 3000 μm.

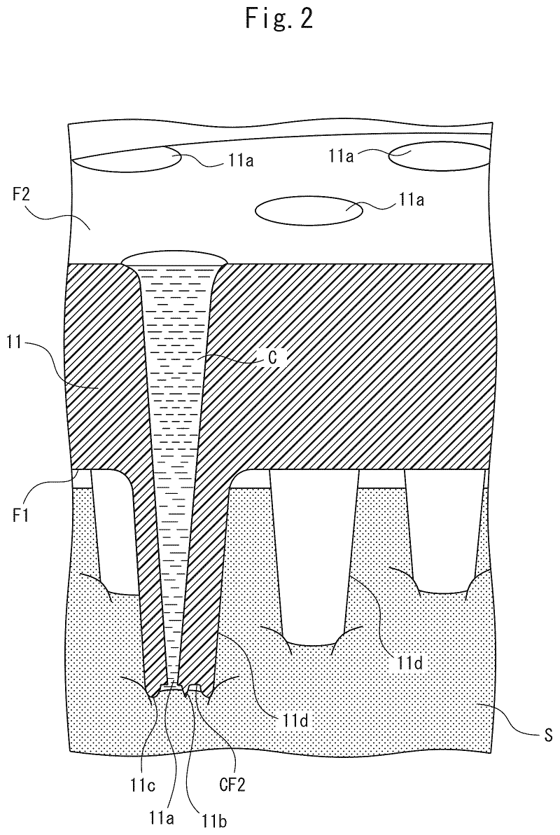

FIG. 2 is a schematic view showing a portion of another embodiment of the needle-like projection-including part according to the present invention.

As shown in FIG. 2, a portion of the needle-like projection-including part comprises a plate 11; a plurality of base portions 11d protruding from one face of the plate 11; a plurality of fine needle-like projections 11b protruding from a front-end face of each of the plurality of base portions 11d; and a lip portion 11c formed on an outer edge of the front-end face of each base portion 11d. In addition, in the plate 11 and the plurality of base portions 11d, a discharge hole 11a penetrating vertically from the other face of the plate 11 to the front-end face of a base portion 11d is formed.

As shown in FIG. 2, when the composition C of the present invention is discharged from the discharge hole 11a, the composition C of the present invention can spread within a space formed between the target object S, a lip portion 11c, and the front-end face of a base portion 11d, and the composition C of the present invention can enter into the target object S via a hole opened by a needle-like projection 11b.

As shown in FIG. 2, the composition C of the present invention can be spread over the target object S on the inner peripheral side of a lip portion 11c on the contact face CF2 of a base portion 11d while penetrating into the inside of the target object S from a hole opened by a needle-like projection 11b. Therefore, the composition C of the present invention can penetrate from both the outside and inside of the target object S.

In the present invention, since a liquid pool can be formed on the front-end side of a base portion 11d, the composition C of the present invention can be spread in a region on the inside of the lip portion 11c of each of the base portions 11d. Therefore, for example, even when the needle-like projection-including part is large and the plate 11 has a large area, the composition C of the present invention can be applied in a pinpoint manner. Further, in the present invention, due to the step between the base portion 11d and one face F1 of the plate 11, the lip portion 11c connected to the base portion 11d can press and spread the target object S in a narrow area to allow a needle-like projection 11b to puncture the target object S, and thus even if the surface of the target object S is uneven or if the target object S is soft, the needle-like projection 11b can easily pierce the target object S, allowing the composition C of the present invention to be more reliably applied from the outside and inside. Furthermore, in the present invention, the lip portion 11c is brought into contact with the target object S regardless of whether the needle-like projection 11b has punctured the target object S, thus allowing injection pressure (resistance) to be applied uniformly to the region on the inner peripheral side of the lip portion 11c, which is the entire front-end portion. As a result, there is an advantage that injectability can be reliably increased. Moreover, in the present invention, since the composition C of the present invention can be transferred more efficiently from the center to the outside in the gap space between the plate 11 and a round plate of the top surface of a container, a band-shaped recessed portion may be formed on the other face F2 of the plate 11, as a flow path along the shortest route for a liquid to flow from the center to the plurality of discharge holes 11a. A plurality of needle-like projections 11b and/or a plurality of discharge holes 11a may be provided in one base portion 11d. In that case, in each of the base portions 11d, the composition C of the present invention pushed out from the plurality of discharge holes 11a can be spread over the target object S on the inner peripheral sides of the lip portions 11c on the contact faces CF2 of the base portions 11d while penetrating into the inside of the target object S from holes opened by the plurality of needle-like projections 11b.

A portion of the needle-like projection-including part shown in FIG. 2 and the entirety (not illustrated) thereof may have various aspects and modifications, in the same manner as the needle-like projection-including part 100 described above.

In the cosmetic kit of the present invention, the cosmetic instrument, in addition to the needle-like projection-including part described above, may further comprise a fixing device to which the needle-like projection-including part can be attached. In addition, the fixing device can store the composition of the present invention.

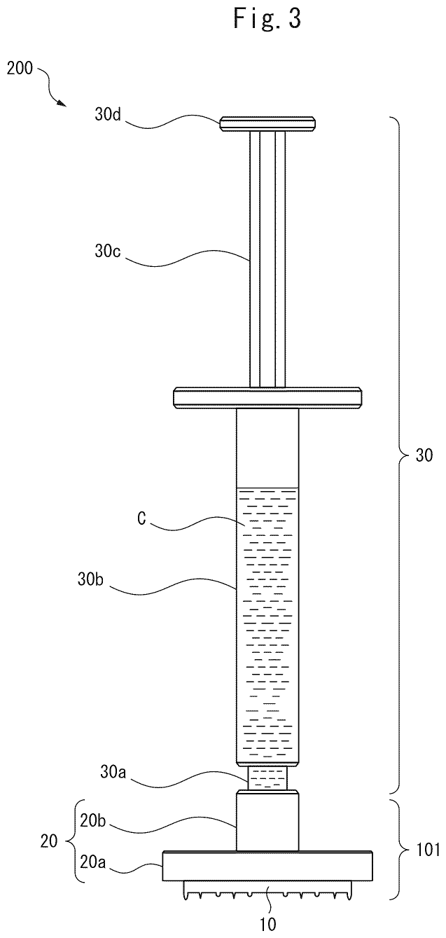

FIG. 3 is a schematic view showing one embodiment of a cosmetic instrument comprising a needle-like projection-including part and a fixing device.

As shown in FIG. 3, a cosmetic instrument 200 comprises a needle-like projection-including part 101 and a syringe 30 as a fixing device to which the needle-like projection-including part 101 is attached. The needle-like projection-including part 101 comprises an attaching frame 20 and a plate 10. The attaching frame 20 comprises a plate-supporting portion 20a and an attaching barrel 20b. The plate 10 comprises a discharge hole (not illustrated) that penetrates vertically and one or a plurality of fine needle-like projections protruding from one face of the plate 10; and a lip portion formed on an outer edge of one face of the plate 10. The syringe 30 comprises a syringe barrel (body) 30b and a pusher 30c. The needle-like projection-including part 101 is attached from the attaching barrel 20b side of the attaching frame 20 to the syringe barrel tip 30a side of the syringe 30. The syringe barrel 30b stores the composition C of the present invention, and the pusher 30c is inserted therein. The pusher 30c is pressed by a pressing piece 30d at the rear end thereof, and is thereby transferred inside the syringe barrel 30b, allowing the composition C of the present invention to be released within the needle-like projection-including part 101 and penetrate into a target object (not illustrated). In the present invention, the fixing device can utilize a syringe having a volume of, for example, 0.5 mL or more, 1.0 mL or more, 2.5 mL or more, 5 mL or more, or 10 mL or more. In addition, the fixing device can utilize a syringe having a volume of, for example, 25 mL or less, 20 mL or less, 15 mL or less, or 10 mL or less. Further, depending on the specific use configuration of the needle-like projection-including part, the syringe may have a larger or smaller volume.

In FIG. 3, the fixing device is illustrated in the shape of a syringe 30, but may be modified to have another shape as long as the effect of the present invention is not impaired.

### <<Cosmetic Method>>

The present invention also provides a cosmetic method. The cosmetic method of the present invention is
a cosmetic method using a needle-type cosmetic instrument and a cosmetic composition for needle-type cosmetic instruments,
wherein the cosmetic composition for needle-type cosmetic instruments has a dynamic surface tension at a temperature of 25±1° C., measured by the Wilhelmy method, of 50 mN/m or less.

The “cosmetic composition for needle-type cosmetic instruments” used in the cosmetic method of the present invention may be the same as the composition of the present invention described above, and thus the description thereof is omitted. In addition, the “needle-type cosmetic instrument” used in the cosmetic method of the present invention is not particularly limited and, for example, the cosmetic instrument (device) disclosed in PTL 1 is preferably used. As a specific example thereof, the description of the cosmetic instrument of the present invention described above may be referenced. Thus, the cosmetic method of the present invention may be a method using the cosmetic kit of the present invention described above.

The cosmetic method of the present invention is strictly for cosmetic purposes, and is not a so-called “medical act” such as a method of surgery on humans or a method for treating a disease of humans.

In the present invention, the skin may not only be the skin of a human, but also the skin of an animal or the surface of a stem, trunk, or leaf of a plant.

### EXAMPLES

The present invention will be further described in detail with reference to the Examples below. However, the present invention is not limited thereto.

### Examples 1 and 2 and Comparative Examples 1 to 4

Based on the formulations in Table 1, the compositions of Examples 1 and 2 and Comparative Examples 1 to 4 were prepared. The compositions of Comparative Example 1, Example 1, and Example 2 were identical to the compositions of Comparative Example 2, Comparative Example 3, and Comparative Example 4, respectively. However, the compositions of Comparative Example 1, Example 1, and Example 2 were used as cosmetic compositions for needle-type cosmetic instruments, while the compositions of Comparative Example 2, Comparative Example 3, and Comparative Example 4 were not used as cosmetic compositions for needle-type cosmetic instruments, and were used as ordinary cosmetic compositions.

Dynamic surface tension of each of the prepared compositions was measured at a temperature of 25±1° C. using a DCAT21 device manufactured by DataPhysics, and an analysis was carried out with the accompanying software SCAT. The resulting dynamic surface tension values are shown in Table 1.

Penetration was examined when each composition was applied to excised human skin (commercial product) via a needle-type cosmetic instrument and when applied directly to the skin without the use of a needle-type cosmetic instrument. More specifically, using a texture analyzer, a load of 1000 gf was applied perpendicularly to the stratum corneum surface of the excised human skin to press a microneedle plate 50 times, after which various compositions containing a fluorescent dye (fluorescein sodium) were applied and left to stand under the condition of 37° C. for 15 minutes. Next, the surface of the skin at the site where each composition had been applied was cleaned, and a portion of the corresponding skin was cut into a circular shape having a diameter of 8 mm with a skin punch. The resulting skin was placed in an appropriate amount of extraction solvent and subjected to an ultrasonic treatment to extract the fluorescent dye. Fluorescence intensity of the extracted sample was then measured with a spectrophotometer. As the needle-type cosmetic instrument, a cosmetic instrument comprising a needle-like projection-including part as shown in FIG. 12 of PTL 1 was used.

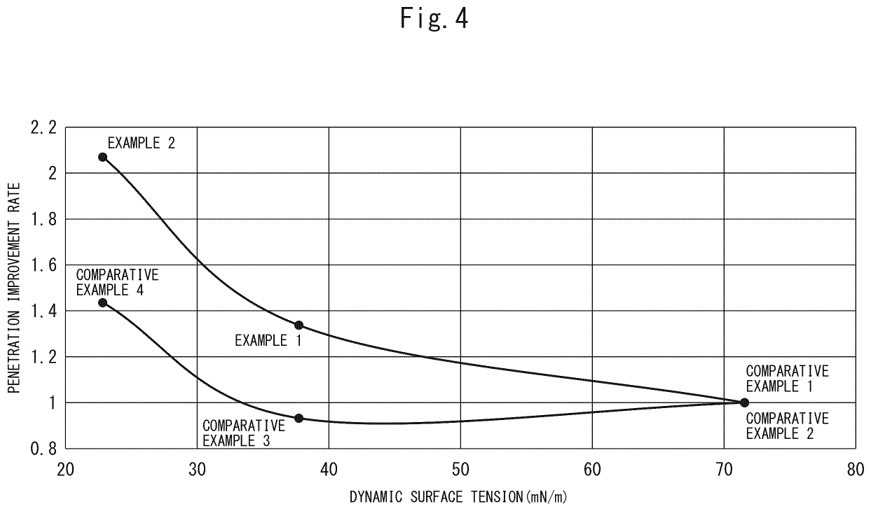

Measurement results (fluorescence intensities) were evaluated as the penetration effectiveness of each composition. The result of the composition of Comparative Example 2, which did not involve a needle-type cosmetic instrument, was set as “1” (reference) when compared with the results of the other compositions, and penetration improvement rates of the other compositions were determined. Results are shown in Table 1 and FIG. 4.

TABLE 1
(Examples 1 and and Comparative 1 to 4)

| | Comparative | Comparative | Example | Comparative | Example | Comparative |
|---|---|---|---|---|---|---|
| Constituent component | Example 1 | Example 2 | 1 | Example 3 | 2 | Example 4 |
| Water | The balance | The balance | The balance | The balance | The balance | The balance |
| Ethanol | — | — | 5.0 | 5.0 | 5.0 | 5.0 |
| Glycerin | — | — | 5.0 | 5.0 | 5.0 | 5.0 |
| Citric acid | 0.01 | 0.01 | 0.01 | 0.01 | 0.01 | 0.01 |
| Sodium citrate | 0.09 | 0.09 | 0.09 | 0.09 | 0.09 | 0.09 |
| Phenoxyethanol | — | — | 0.3 | 0.3 | 0.3 | 0.3 |
| Fluorescein sodium | 0.02 | 0.02 | 0.02 | 0.02 | 0.02 | 0.02 |
| (fluorescent component) | | | | | | |
| Potassium cocoyl glutamate | — | — | 0.014 | 0.014 | 0.1 | 0.1 |
| Total | 100 | 100 | 100 | 100 | 100 | 100 |
| Dynamic surface tension (mN/m) | 71.57 | 71.57 | 37.72 | 37.72 | 22.82 | 22.82 |
| Needle-type cosmetic instrument | Used | Not used | Used | Not used | Used | Not used |
| Penetration improvement rate | 1.00 | 1.00 | 1.34 | 0.93 | 2.07 | 1.44 |
| | | (reference) | | | | |

*In Table 1, numerical value corresponding to each constituent component in the rows up to the row “Total” indicates blending amount for each component, expressed in units of “% by mass”. The same applies to the tables described below.

As is clear from the results in Table 1 and FIG. 4, penetrations of the compositions of Comparative Examples 1 and 2, which had relatively high dynamic surface tensions, were found to be similar regardless of whether a needle-type cosmetic instrument was used or not.

From the results for the compositions of Examples 1 and 2 and Comparative Examples 3 and 4, which had relatively low dynamic surface tensions, penetration was found to be significantly improved by the use of a needle-type cosmetic instrument.

### Examples 3 and 4 and Comparative Example 5

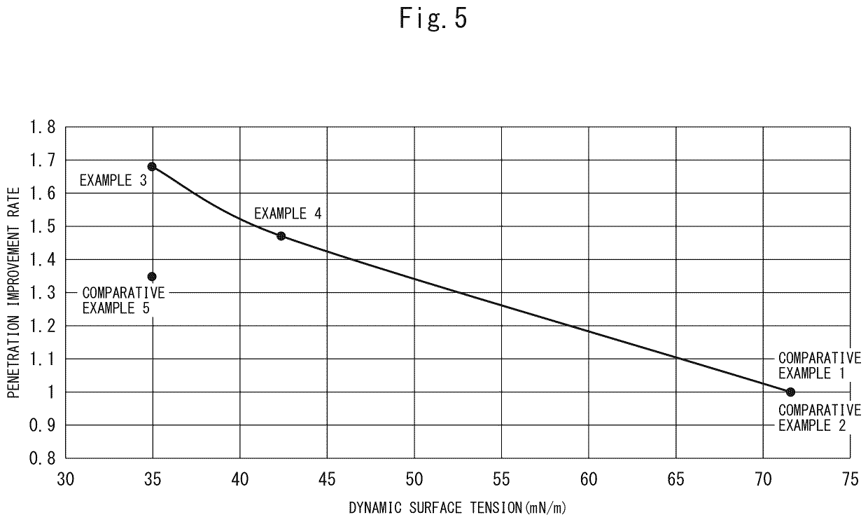

Based on the formulations in Table 2, the compositions of the Examples and Comparative Examples were prepared, and dynamic surface tension and penetration improvement rate were examined in the same manner as above. Results are shown in Table 2 and FIG. 5. Results are shown in Table 2 and FIG. 5.

TABLE 2
(Examples 3 and 4 and Comparative Examples 1, 2, and 5)

| | Comparative | Comparative | | Comparative | |
|---|---|---|---|---|---|
| Constituent component | Example 1 | Example 2 | Example 3 | Example 5 | Example 4 |
| Water | The balance | The balance | The balance | The balance | The balance |
| Ethanol | — | — | 5.0 | 5.0 | 5.0 |
| Glycerin | — | — | 5.0 | 5.0 | 5.0 |
| Citric acid | 0.01 | 0.01 | 0.01 | 0.01 | 0.01 |
| Sodium citrate | 0.09 | 0.09 | 0.09 | 0.09 | 0.09 |
| Phenoxyethanol | — | — | 0.3 | 0.3 | 0.3 |
| Fluorescein sodium | 0.02 | 0.02 | 0.02 | 0.02 | 0.02 |
| (fluorescent component) | | | | | |
| Sodium dilauroyl glutamate lysine | — | — | 0.3 | 0.3 | — |
| Total | 100 | 100 | 100 | 100 | 100 |
| Dynamic surface tension (mN/m) | 71.57 | 71.57 | 34.95 | 34.95 | 42.36 |
| Needle-type cosmetic instrument | Used | Not used | Used | Not used | Used |
| Penetration improvement rate | 1.00 | 1.00 | 1.68 | 1.35 | 1.47 |
| | | (reference) | | | |

As is clear from the results in Table 2 and FIG. 5, penetrations of the compositions of Comparative Examples 1 and 2, which had relatively high dynamic surface tensions, were found to be similar regardless of whether a needle-type cosmetic instrument was used or not.

From the results for the compositions of Example 3 and Comparative Example 5, which had relatively low dynamic surface tensions, penetration was found to be significantly improved by the use of a needle-type cosmetic instrument. Likewise, from the results for the composition of Example 4, which had a relatively low dynamic surface tension, penetration was found to be significantly improved by the use of a needle-type cosmetic instrument.

### Formulation Examples

In the following, Formulation Examples of the composition of the present invention are recited by way of example. The present invention is not limited in any way by the following Formulation Examples.

### Formulation Example 1

TABLE 3
Formulation Example 1 (unit is “% by mass”)

| Water | The balance |
|---|---|
| Ethanol | 5 |
| Glycerin | 5 |
| Dipropylene glycol | 8 |
| Xylitol | 1 |
| Sodium dilauroyl glutamate lysine | 0.02 |
| Sodium hyaluronate | 0.05 |
| Nicotinamide | 5 |
| Dipotassium glycyrrhizate | 0.2 |
| Phellodendron amurense bark extract | 0.01 |
| Trifolium pratense flower extract | 0.01 |
| Citric acid | Appropriate amount |
| Sodium citrate | Appropriate amount |
| EDTA-2Na | Appropriate amount |
| Sodium pyrosulfite | Appropriate amount |
| Phenoxyethanol | Appropriate amount |
| Total | 100 |

### Formulation Example 2

TABLE 4
Formulation Example 2 (unit is “% by mass”)

| Water | The balance |
|---|---|
| Ethanol | 5 |
| Glycerin | 5 |
| Dipropylene glycol | 7 |
| 1,3-Butylene glycol | 1 |
| Sodium hyaluronate | 0.1 |
| Xylitol | 3 |
| Isostearyl alcohol | 0.13 |
| Hydrogenated polydecene | 0.25 |
| Isostearic acid | 0.13 |
| PEG/PPG-17/4 dimethyl ether | 0.1 |
| Sorbitan sesquiisostearate | 0.02 |
| PEG-30 phytosterol | 0.14 |
| Potassium cocoyl glutamate | 0.01 |
| Nicotinamide | 3 |
| Potassium salt of 4-methoxysalicylic acid | 1 |
| Tocopherol acetate | 0.1 |
| Dipotassium glycyrrhizate | 0.5 |
| 2-O-Ethyl ascorbic acid | 0.1 |
| α-Glucan oligosaccharide | 0.01 |
| Bupleurum root extract | 0.01 |
| Citric acid | Appropriate amount |
| Sodium citrate | Appropriate amount |
| Potassium hydroxide | Appropriate amount |
| EDTA-2Na | Appropriate amount |
| Phenoxyethanol | Appropriate amount |
| Total | 100 |

### Formulation Example 3

TABLE 5
Formulation Example 3 (unit is “% by mass”)

| Water | The balance |
|---|---|
| Ethanol | 10 |
| Glycerin | 8 |
| Dipropylene glycol | 5 |
| 1,3-Butylene glycol | 5 |
| Erythritol | 1 |
| Xanthan gum | 0.01 |
| Isostearyl alcohol | 0.03 |
| Isostearic acid | 0.03 |
| PEG/PPG-14/7 dimethyl ether | 0.13 |
| PEG-60 hydrogenated castor oil | 0.08 |
| Polyglyceryl-2 diisostearate | 0.04 |
| Nicotinamide | 1 |
| Potassium salt of 4-methoxysalicylic acid | 1 |
| Tocopherol acetate | 0.1 |
| Dipotassium glycyrrhizate | 0.5 |
| 2-O-Ethyl ascorbic acid | 0.1 |
| Lactobacillus/rice ferment | 0.01 |
| Cassia bark extract | 0.01 |
| Citric acid | Appropriate amount |
| Sodium citrate | Appropriate amount |
| Potassium hydroxide | Appropriate amount |
| EDTA-2Na | Appropriate amount |
| Phenoxyethanol | Appropriate amount |
| Total | 100 |

### REFERENCE SIGNS LIST

10, 11 plate
10a, 11a discharge hole
10b, 11b needle-like projection
10c, 11c lip portion
11d base portion
20 attaching frame
20a plate-supporting portion
20b attaching barrel
30 syringe
30a syringe barrel tip
30b syringe barrel
30c pusher
30d pressing piece
100, 101 needle-like projection-including part
200 cosmetic instrument
C composition of present invention
S target object (skin)

1. A cosmetic composition for needle-type cosmetic instruments, wherein a dynamic surface tension at a temperature of 25±1° C., measured by Wilhelmy method, is 50 mN/m or less.

2. The composition according to claim 1, comprising a dynamic surface tension-reducing component selected from a group consisting of amino acid-based substances, surfactants, lower alcohols, higher alcohols, polyhydric alcohols, aromatic alcohols, silicones, fluorine compounds, saccharides, terpenes, acrylic compounds, polyethylene glycol, polyvinyl alcohol, and combinations thereof.

3. The composition according to claim 2, wherein a content of the dynamic surface tension-reducing component is an amount that reduces the dynamic surface tension by 20 mN/m or more.

4. The composition according to claim 2, wherein the amino acid-based substance comprises sodium dilauroyl glutamate lysine and/or potassium cocoyl glutamate.

5. A cosmetic kit comprising a needle-type cosmetic instrument and the cosmetic composition for needle-type cosmetic instruments according to claim 1.

6. A cosmetic method using a needle-type cosmetic instrument and the cosmetic composition for needle-type cosmetic instruments according to claim 1.

7. The cosmetic composition for needle-type cosmetic instruments according to claim 1,
wherein the needle-type cosmetic instrument comprises a needle-like projection-including part that injects the cosmetic composition for needle-type cosmetic instruments into a target object.

8. The kit according to claim 5,
wherein the needle-type cosmetic instrument comprises a needle-like projection-including part that injects the cosmetic composition for needle-type cosmetic instruments into a target object.
